(19) United States
(12) Patent Application Publication
(10) Pub. No.: US 2024/0358348 A1
(43) Pub. Date: Oct. 31, 2024

(54) CATHETER

(71) Applicant: ASAHI INTECC CO., LTD., Seto-shi (JP)

(72) Inventors: Yoshiki KATO, Seto-shi (JP); Hirotomo SHIMIZU, Seto-shi (JP); Yukiko HASE, Seto-shi (JP)

(73) Assignee: ASAHI INTECC CO., LTD., Seto-shi (JP)

(21) Appl. No.: 18/768,032

(22) Filed: Jul. 10, 2024

Related U.S. Application Data
(63) Continuation of application No. PCT/JP2022/048249, filed on Dec. 27, 2022.

(30) Foreign Application Priority Data
Jan. 13, 2022 (JP) 2022-003556

(51) Int. Cl. A61B 8/12; A61B 8/00; A61M 25/09

(52) U.S. Cl. CPC A61B 8/12; A61B 8/4254; A61B 8/445; A61M 25/09

(57) ABSTRACT

A catheter includes a shaft part having a first shaft and a second shaft. The first shaft is a cylindrical member having a guide wire lumen into which a guide wire is inserted. The second shaft is a cylindrical member having an imaging lumen into which an imaging device for acquiring an image of an interior of a living body lumen is inserted, and is disposed alongside the first shaft. In a first region from a first position at a distal end portion of the shaft part to a second position further on a proximal end side than the first position, the second shaft is not joined to the first shaft. In a second region that is continuous with the first region and that is further on the proximal end side than the first region, the second shaft is joined to the first shaft.

### CROSS-REFERENCE TO RELATED APPLICATIONS

This application is a continuation application of International Application No. PCT/JP2022/048249, filed Dec. 27, 2022, which claims priority to Japanese Patent Application No. 2022-003556, filed Jan. 13, 2022. The contents of these applications are incorporated herein by reference in their entirety.

### TECHNICAL FIELD

The technique disclosed herein relates to a catheter.

### BACKGROUND ART

In the background art, in order to acquire a clearer image in intravascular ultrasound (IVUS), a configuration in which a large opening (a portion where a resin material is not present) is provided at a position facing a transducer in a first shaft having a guide wire lumen is known (for example, see Patent Literature 1).

### CITATION LIST

### Patent Literature

Patent Literature 1: Japanese Unexamined Patent Application Publication No. 2004-97286

### SUMMARY

### Technical Problems

In conventional IVUS catheters, a second shaft is joined to the first shaft along its entire length. That is, the second shaft is joined to the first shaft even at the position where the transducer of an IVUS device is disposed. Therefore, transmission and reception of ultrasonic waves by the transducer of the IVUS device inserted into the IVUS lumen of the second shaft are hindered at the joint part, and acquisition of a clear image by the IVUS device is hindered. To be more specific, when the first shaft and the second shaft are joined to each other with an adhesive, the presence of the adhesive hinders transmission and reception of ultrasonic waves on the first shaft side by the transducer. In a case where the first shaft and the second shaft are joined to each other by welding, distortion occurs in the shafts due to the welding, and a range in which transmission and reception of ultrasonic waves on the first shaft side by the transducer are hindered increases. Thus, there is room for improvement in the conventional IVUS catheters in terms of acquiring a clear image with the IVUS device.

Such a problem is not limited to an IVUS catheter having an IVUS lumen into which an IVUS device is inserted, and is common to catheters having an imaging lumen into which an imaging device for acquiring an image of the interior of a living body lumen is inserted.

### Solutions to Problems

A catheter disclosed herein includes a shaft part having a first shaft and a second shaft. The first shaft is a cylindrical member having a guide wire lumen into which a guide wire is inserted. The second shaft is a cylindrical member having an imaging lumen into which an imaging device for acquiring an image of an interior of a living body lumen is inserted and is disposed alongside the first shaft. In a first region from a first position at a distal end portion of the shaft part to a second position that is further on a proximal end side than the first position, the second shaft is not joined to the first shaft. In a second region that is continuous with the first region and further on the proximal end side than the first region, the second shaft is joined to the first shaft.

### BRIEF DESCRIPTION OF DRAWINGS

FIG. 1 is an explanatory view schematically illustrating a configuration of a recanalization catheter system according to a first embodiment.

FIG. 2 is an explanatory view illustrating a configuration of an IVUS catheter.

FIG. 3 is an explanatory view illustrating a configuration of the IVUS catheter.

FIG. 4 is an explanatory view illustrating a configuration of the IVUS catheter.

FIG. 5 is an explanatory view illustrating a configuration of the IVUS catheter.

FIG. 6 is an explanatory view illustrating a configuration of the IVUS catheter.

FIG. 7 is an explanatory view illustrating an example of a use mode of the IVUS catheter.

FIG. 8 is an explanatory view illustrating another example of the use mode of the IVUS catheter.

FIG. 9 is an explanatory view schematically illustrating a configuration of an IVUS device.

FIG. 10 is an explanatory view illustrating an example of a method of using the recanalization catheter system.

FIG. 11 is an explanatory view illustrating an example of a method of using the recanalization catheter system.

FIG. 12 is an explanatory view schematically illustrating a configuration of an IVUS catheter according to a second embodiment.

FIG. 13 is an explanatory view schematically illustrating a configuration of an IVUS catheter according to a third embodiment.

### DETAILED DESCRIPTION

### A. First Embodiment

### A-1. Configuration of Recanalization Catheter System 10

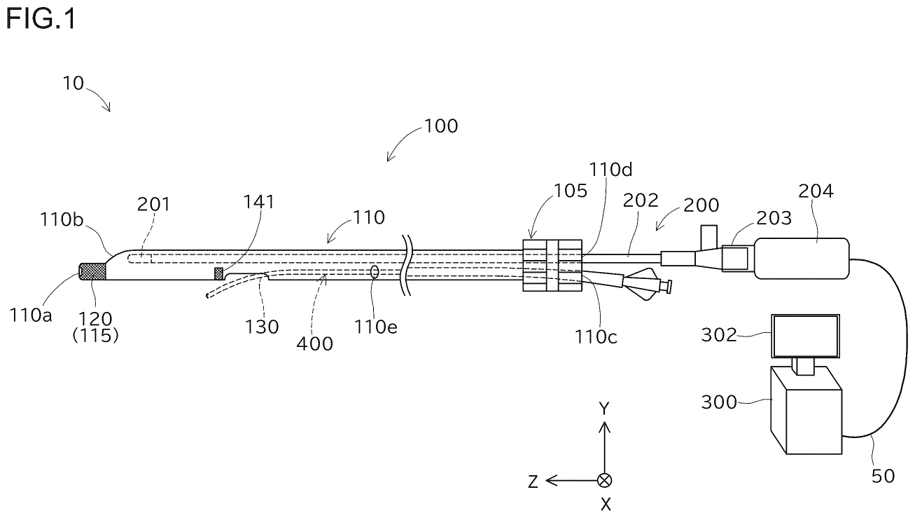

FIG. 1 is an explanatory view schematically illustrating a configuration of a recanalization catheter system 10 according to a first embodiment. The recanalization catheter system 10 is used, for example, when a chronic total occlusion (CTO) generated in a blood vessel is treated by an antegrade approach. The recanalization catheter system 10 includes an IVUS catheter 100, an IVUS device 200, and an imaging console 300.

An entirety of the recanalization catheter system 10 is indicated by break lines in FIG. 1 for ease of illustration. In FIG. 1, mutually orthogonal XYZ axes are illustrated. In a device such as the IVUS catheter 100, a Z-axis positive direction side is a distal end side (distal side) to be inserted into a body, and a Z-axis negative direction side is a proximal end side (near side) to be operated by a professional such as a doctor. In FIG. 1, the IVUS catheter 100 is illustrated in a substantially linear state parallel to the Z-axis direction, but the IVUS catheter 100 has flexibility to the extent that it can be curved. These points are also the same in the subsequent drawings. Herein, in the recanalization catheter system 10 and the component member thereof, the terminal of a distal end side is referred to as “distal end”, the distal end and the vicinity thereof is referred to as “distal end portion”, the terminal of a proximal end side is referred to as “proximal end”, and the proximal end and the vicinity thereof is referred to as “proximal end portion”.

### Configuration of IVUS Catheter 100

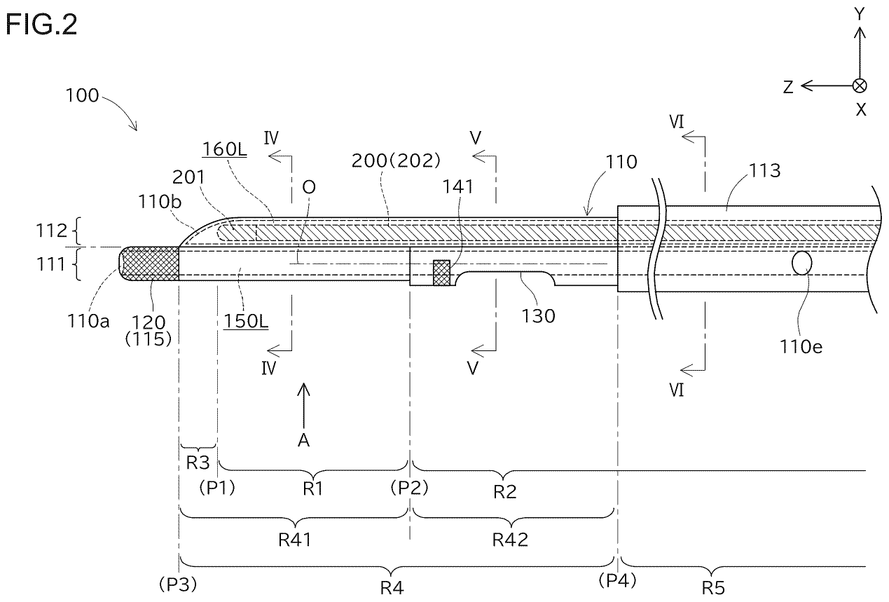

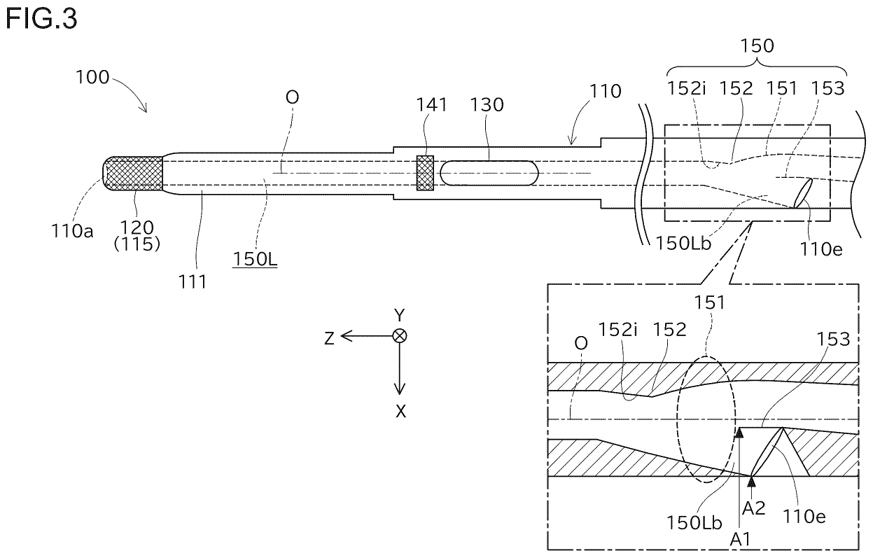

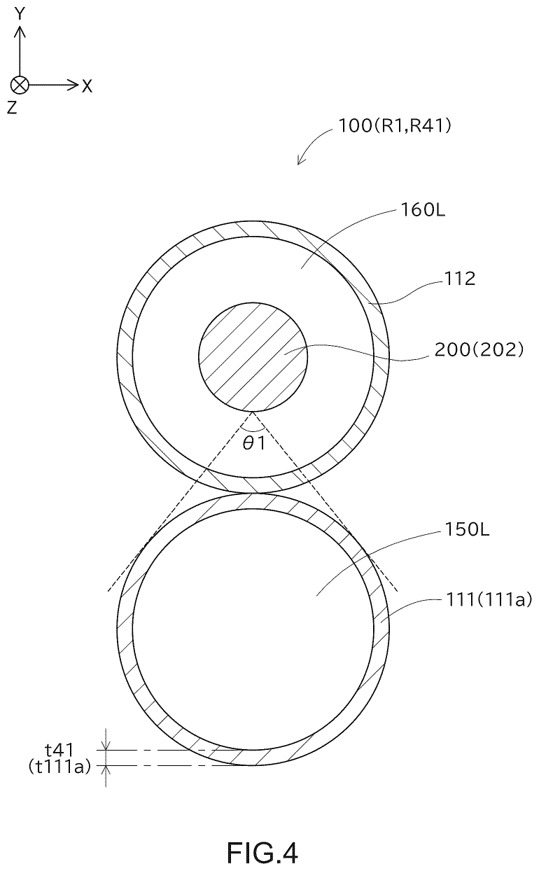

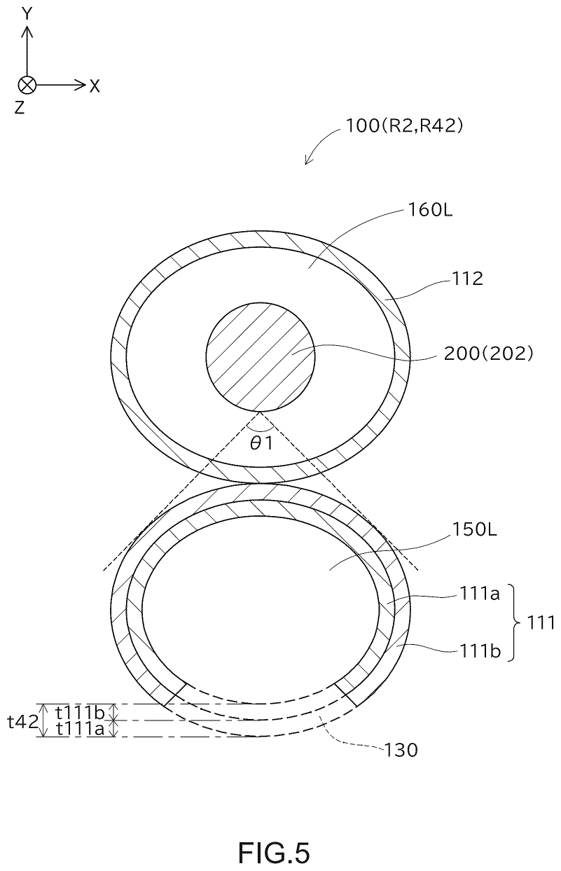

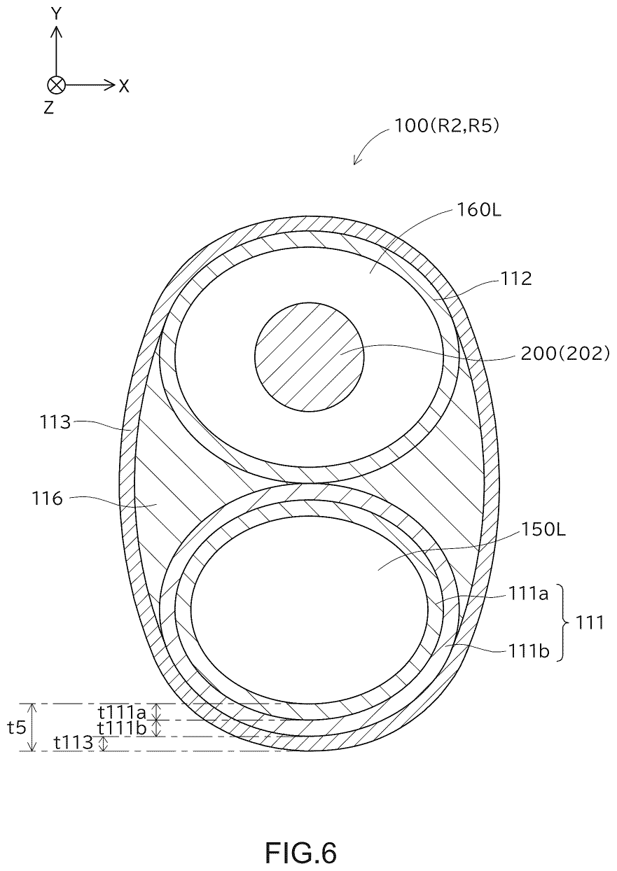

FIG. 2 to FIG. 6 are explanatory views illustrating a configuration of the IVUS catheter 100. FIG. 2 illustrates an enlarged side view of the distal end portion of the IVUS catheter 100, FIG. 3 illustrates an enlarged side view (lower surface) of the IVUS catheter 100 as viewed from direction A in FIG. 2, FIG. 4 illustrates a transverse section configuration of the IVUS catheter 100 taken along line IV-IV in FIG. 2, FIG. 5 illustrates a transverse section configuration of the IVUS catheter 100 taken along line V-V in FIG. 2, and FIG. 6 illustrates a transverse section configuration of the IVUS catheter 100 taken along line VI-VI in FIG. 2.

The IVUS catheter 100 is an elongated medical device used when performing IVUS, which is a technique for acquiring an image of the interior of a living body lumen such as a blood vessel. The IVUS catheter 100 includes an elongated shaft part 110. As illustrated in FIG. 2, the shaft part 110 includes a first inner shaft 111, a second inner shaft 112, and an outer shaft 113. The first inner shaft 111 is an example of a first shaft in the claims, and the second inner shaft 112 is an example of a second shaft in the claims.

The second inner shaft 112 is a substantially cylindrical member having an IVUS lumen 160L into which the IVUS device 200 is inserted. A distal end second opening 110b that allows the IVUS lumen 160L to communicate with the outside is formed at the distal end of the second inner shaft 112, and a proximal end second opening 110d that allows the IVUS lumen 160L to communicate with the outside is formed at the proximal end of the second inner shaft 112 (FIG. 1). The distal end second opening 110b is an opening for discharging a fluid injected into the IVUS lumen 160L from the proximal end second opening 110d. The distal end second opening 110b does not need to be formed at the distal end of the second inner shaft 112, e.g., may be formed at the distal end portion of the second inner shaft 112. The IVUS lumen 160L is an example of an imaging lumen in the claims.

The first inner shaft 111 is a substantially cylindrical member having a guide wire lumen 150L into which a guide wire is inserted. A distal end first opening 110a that allows the guide wire lumen 150L to communicate with the outside is formed at the distal end of the first inner shaft 111, and a proximal end first opening 110c that allows the guide wire lumen 150L to communicate with the outside is formed at the proximal end of the first inner shaft 111 (FIG. 1).

The first inner shaft 111 and the second inner shaft 112 are disposed alongside in a Y-axis direction in a state where the extending directions thereof are parallel to each other. As illustrated in FIG. 2, the most distal end portion of the first inner shaft 111 (hereinafter, referred to as “protruding portion 115”) protrudes toward the distal end side from a third position P3 that is the distal end position of the second inner shaft 112. Therefore, the distal end first opening 110a is positioned further on the distal end side than the distal end second opening 110b. The distal end portion of the second inner shaft 112 has a shape in which the outer diameter gradually decreases from the proximal end side toward the distal end side and the distal end surface is smoothly connected to the protruding portion 115 of the first inner shaft 111.

A distal tip 120 is joined to at least a part of the protruding portion 115 of the first inner shaft 111. The distal tip 120 is formed of, for example, a material having radiopacity. The shape of the distal tip 120 can be freely set, and can be, for example, a substantially cylindrical shape with R at the distal end portion or a substantially truncated conical shape in which the outer diameter gradually decreases from the proximal end side toward the distal end side.

The second inner shaft 112 is joined to the first inner shaft 111 at a part along the extending direction, and is not joined to the first inner shaft 111 at the remaining part. To be more specific, as illustrated in FIG. 2, the second inner shaft 112 is not joined to the first inner shaft 111 in a part on the distal end side of the shaft part 110, specifically, in a first region R1 from a first position Pl positioned on the proximal end side of the distal end of the second inner shaft 112 to a second position P2 positioned further on the proximal end side than the first position P1. The length of the first region R1 along the extending direction of the shaft part 110 may be, for example, from 10 mm to 30 mm. On the other hand, the second inner shaft 112 is joined to the first inner shaft 111 in a second region R2 that is continuous with the first region R1 and that is positioned further on the proximal end side than the first region R1, specifically, in a region from the second position P2 to the proximal end of the second inner shaft 112. The second inner shaft 112 is joined to the first inner shaft 111 in a third region R3 that is continuous with the first region R1 and that is positioned further on the distal end side than the first region R1, specifically, in a region from the first position P1 to the third position P3 that is the distal end of the second inner shaft 112. As described above, in the IVUS catheter 100 of the present embodiment, three portions, that is, a joint part where the second inner shaft 112 is joined to the first inner shaft 111, a non-joint part where the second inner shaft 112 is not joined to the first inner shaft 111, and another joint part where the second inner shaft 112 is joined to the first inner shaft 111 are arranged in order from the distal end side to the proximal end side.

As the joint between the first inner shaft 111 and the second inner shaft 112, joint (welding) between resins by thermal melting or bonding with an insulating adhesive such as an epoxy-based adhesive can be adopted. In the present embodiment, it is assumed that the first inner shaft 111 and the second inner shaft 112 are joined by welding. Therefore, in the first region R1 where the first inner shaft 111 and the second inner shaft 112 are not joined to each other, as illustrated in FIG. 4, the traverse sections of both have a shape close to a perfect circle, whereas in the second region R2 where both are joined to each other, as illustrated in FIGS. 5 and 6, the traverse sections of both have a shape distorted into a flat shape due to welding.

As illustrated in FIGS. 4 to 6, the second inner shaft 112 has a single layer configuration over the entire length. Meanwhile, the first inner shaft 111 has a single layer configuration including only a first layer 111a in the third region R3 and the first region R1, but has a two layer configuration including the first layer 111a and a second layer 111b in the second region R2. In the present embodiment, a wall thickness t111a of the first layer 111a is substantially the same as a wall thickness t111b of the second layer 111b.

As illustrated in FIG. 6, the outer shaft 113 is a substantially elliptic cylindrical member. The first inner shaft 111 and the second inner shaft 112 are accommodated in the inner space of the outer shaft 113. A space around the first inner shaft 111 and the second inner shaft 112 in the inner space of the outer shaft 113 is filled with a filler 116, whereby the first inner shaft 111 and the second inner shaft 112 are fixed. As illustrated in FIG. 2, the distal end of the outer shaft 113 is positioned at a fourth position P4 that is further on the proximal end side than the second position P2. That is, the outer shaft 113 does not cover the first inner shaft 111 and the second inner shaft 112 in a fourth region R4 that is a region from the third position P3 to the fourth position P4, and covers the first inner shaft 111 and the second inner shaft 112 in a fifth region R5 that is continuous with the fourth region R4 and that is positioned further on the proximal end side than the fourth region R4. The fourth region R4 is a region including the first region R1. Hereinafter, in the fourth region R4, a region from the third position P3 to the second position P2 is referred to as a first small region R41, and a region from the second position P2 to the fourth position P4 is referred to as a second small region R42.

In the IVUS catheter 100 according to the present embodiment, the minimum value of the wall thickness of the shaft part 110 at the position of the guide wire lumen 150L varies depending on a position along the extending direction. Here, the minimum value of the wall thickness of the shaft part 110 at the position of the guide wire lumen 150L is the shortest length from the inner peripheral surface of the guide wire lumen 150L to the outer peripheral surface of the shaft part 110 in the transverse section of the shaft part 110 (see FIGS. 4 to 6), and is hereinafter referred to as “minimum wall thickness value of the shaft part on the guide wire lumen side”. To be specific, in the first small region R41 (the third region R3 and the first region R1) of the fourth region R4, as illustrated in FIG. 4, a minimum wall thickness value t41 of the shaft part on the guide wire lumen side coincides with the wall thickness t111a of the first layer 111a of the first inner shaft 111. In the second small region R42 of the fourth region R4, as illustrated in FIG. 5, a minimum wall thickness value t42 of the shaft part on the guide wire lumen side coincides with the sum of the wall thickness t111a of the first layer 111a and the wall thickness t111b of the second layer 111b of the first inner shaft 111. In the fifth region R5, as illustrated in FIG. 6, a minimum wall thickness value t5 of the shaft part on the guide wire lumen side coincides with the sum of the wall thickness t111a of the first layer 111a, the wall thickness t111b of the second layer 111b, and a wall thickness t113 of the outer shaft 113. Therefore, the minimum wall thickness value of the shaft part on the guide wire lumen side increases in the order of the first small region R41, the second small region R42, and the fifth region R5. That is, the minimum wall thickness value of the shaft part on the guide wire lumen side gradually increases from the distal end side toward the proximal end side.

As illustrated in FIGS. 2 and 3, the first inner shaft 111 is formed with a notch 130 that allows the guide wire lumen 150L to communicate with the outside. The notch 130 is positioned in the second region R2 (more specifically, the second small region R42) described above. The notch 130 is formed in the side surface of the first inner shaft 111 at a position opposite to the IVUS lumen 160L with respect to a center axis O of the guide wire lumen 150L. As illustrated in FIG. 3, the notch 130 has a substantially elliptical shape having a long axis in the extending direction of the first inner shaft 111 (Z-axis direction) when viewed in the direction in which the guide wire lumen 150L and the IVUS lumen 160L are arranged (Y-axis direction). A marker 141 formed of, for example, a material having radiopacity is provided in the vicinity of the notch 130 (on the distal end side of the notch 130 in the present embodiment).

The IVUS lumen 160L formed in the second inner shaft 112 extends from the distal end to the proximal end of the second inner shaft 112 along the center axis of the second inner shaft 112. Meanwhile, as illustrated in FIG. 3, the guide wire lumen 150L formed in the first inner shaft 111 similarly extends from the distal end to the proximal end of the first inner shaft 111 along the center axis of the first inner shaft 111, but branches at an intermediate position (for example, a position separated from the distal end by approximately 200 mm to 400 mm) and communicates with the outside via a port 110e formed in the side surface of the shaft part 110. Hereinafter, a lumen branching from the guide wire lumen 150L and connected to the port 110e is referred to as a “branching lumen 150Lb”. In the shaft part 110, a part around a connection portion between the guide wire lumen 150L and the branching lumen 150Lb is referred to as a “branching part 150”.

The branching part 150 includes a large diameter portion 151, a raised portion 152, and a boundary wall 153. The large diameter portion 151 is a portion having a larger inner diameter than other portions of the guide wire lumen 150L. The raised portion 152 is a portion where a part of an inner peripheral surface 152i defining the guide wire lumen 150L in the inner peripheral surface of the branching part 150 is raised. The raised portion 152 is provided further on the distal end side than the large diameter portion 151 in the inner peripheral surface 152i of the branching part 150. At the raised portion 152, the inner peripheral surface 152i of the branching part 150 is raised toward the side where the branching lumen 150Lb extends. The boundary wall 153 is a part of the shaft part 110 provided further on the proximal end side than the large diameter portion 151, and is a portion that separates the guide wire lumen 150L from the branching lumen 150Lb. A distal end A1 of the boundary wall 153 is positioned further on the distal end side than a distal end A2 of the port 110e.

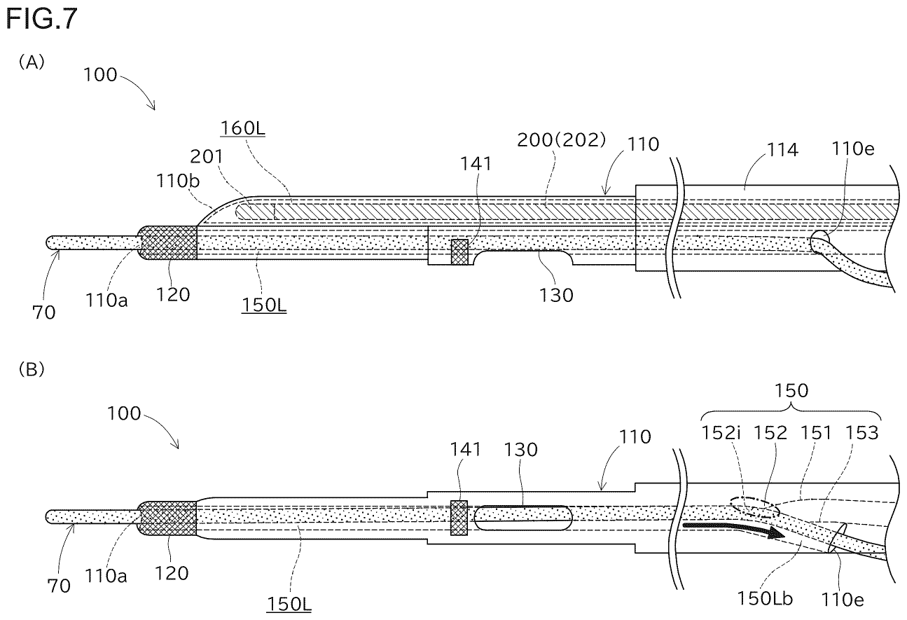

FIG. 7 is an explanatory view illustrating an example of a use mode of the IVUS catheter 100. (A) of FIG. 7 illustrates a side view of the IVUS catheter 100 viewed from the same direction as in FIG. 2, and (B) of FIG. 7 illustrates a side view (lower surface) of the IVUS catheter 100 viewed from the same direction as in FIG. 3. FIG. 7 illustrates a use mode of the IVUS catheter 100 in a case where the delivery guide wire 70 used for delivering the IVUS catheter 100 is inserted into the guide wire lumen 150L from the distal end first opening 110a and advances from the distal end side toward the proximal end side in the guide wire lumen 150L (in other words, a case where the IVUS catheter 100 is used as a rapid exchange type (Rx type) catheter, hereinafter referred to as a “first case”).

In the first case, an operator inserts the proximal end portion of the delivery guide wire 70 into the guide wire lumen 150L from the distal end first opening 110a of the IVUS catheter 100, and pulls the proximal end portion of the delivery guide wire 70 to the outside from the port 110e via the branching lumen 150 Lb. In doing so, the proximal end portion of the delivery guide wire 70 comes into contact with the raised portion 152, and is thereby naturally guided toward the branching lumen 150Lb (in the direction of a thick arrow).

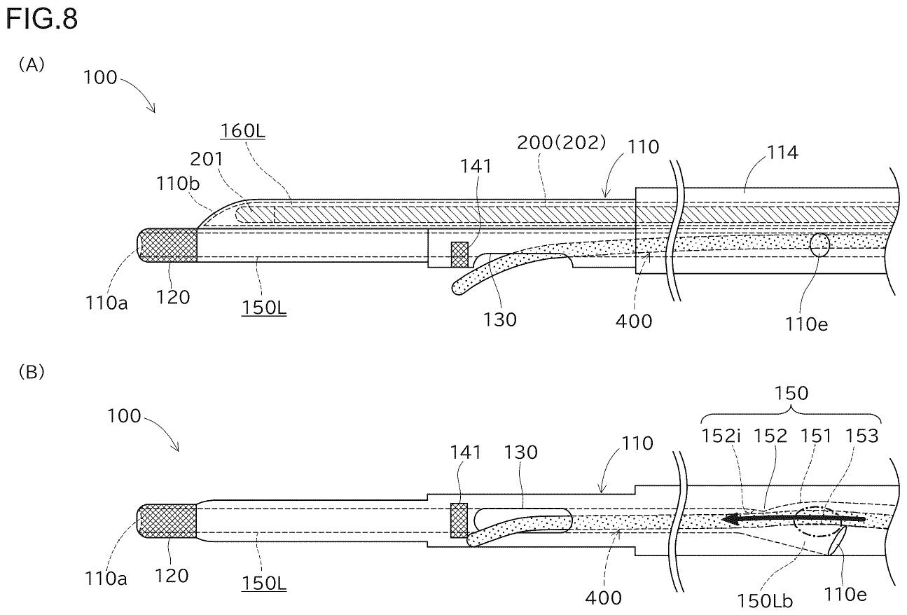

FIG. 8 is an explanatory view illustrating another example of the use mode of the IVUS catheter 100. (A) of FIG. 8 illustrates a side view of the IVUS catheter 100 viewed from the same direction as in FIG. 2, and (B) of FIG. 8 illustrates a side view (lower surface) of the IVUS catheter 100 viewed from the same direction as in FIG. 3. FIG. 8 illustrates a use mode of the IVUS catheter 100 in a case where a penetration guide wire 400 used for penetrating a CTO lesion is inserted into the guide wire lumen 150L from the proximal end first opening 110c (FIG. 1) and advances from the proximal end side toward the distal end side in the guide wire lumen 150L (in other words, a case where the IVUS catheter 100 is used as an over-the-wire type (OTW-type) catheter, hereinafter referred to as a “second case”).

In the second case, the operator inserts the distal end portion of the penetration guide wire 400 into the guide wire lumen 150L from the proximal end first opening 110c, causes the distal end portion to advance straight so as to pass through the branching part 150 (without causing the penetration guide wire 400 to enter the branching lumen 150Lb), and pulls the distal end portion to the outside from the notch 130. In doing so, the distal end portion of the penetration guide wire 400 comes into contact with the boundary wall 153, and is thereby guided to naturally pass through the branching part 150 (in the direction of a thick arrow).

Returning to FIG. 1, the IVUS catheter 100 further includes an adjuster 105. The adjuster 105 is a portion that performs an operation for advancing or retracting the IVUS device 200 in the IVUS lumen 160L. The adjuster 105 includes, for example, a dial that can be operated by the operator, and when the dial is rotated, the IVUS device 200 moves forward or backward.

The outer shaft 113, the first inner shaft 111, the second inner shaft 112, the filler 116, and the adjuster 105 may be formed of a known material, for example, nylon resin such as polyamide, polyolefin such as polyethylene, polypropylene, and ethylene-propylene copolymers, polyester such as polyethylene terephthalate, thermoplastic resin such as polyvinyl chloride, ethylene-vinyl acetate copolymers, cross-linked ethylene-vinyl acetate copolymers, and polyurethane, polyamide elastomer, polyolefin elastomer, polyurethane elastomer, silicone rubber, and latex rubber. The outer shaft 113, the first inner shaft 111, the second inner shaft 112, the filler 116, and the adjuster 105 may be formed of the same material, or at least some or all of them may be formed of a material different from the others. Regarding the outer shaft 113, the first inner shaft 111, the second inner shaft 112, and the filler 116, at least a part positioned in the vicinity of the notch 130 may be formed of a resin having a small difference in acoustic impedance from a biological tissue, for example, polyethylene.

### Configuration of the IVUS Device 200 or the Like

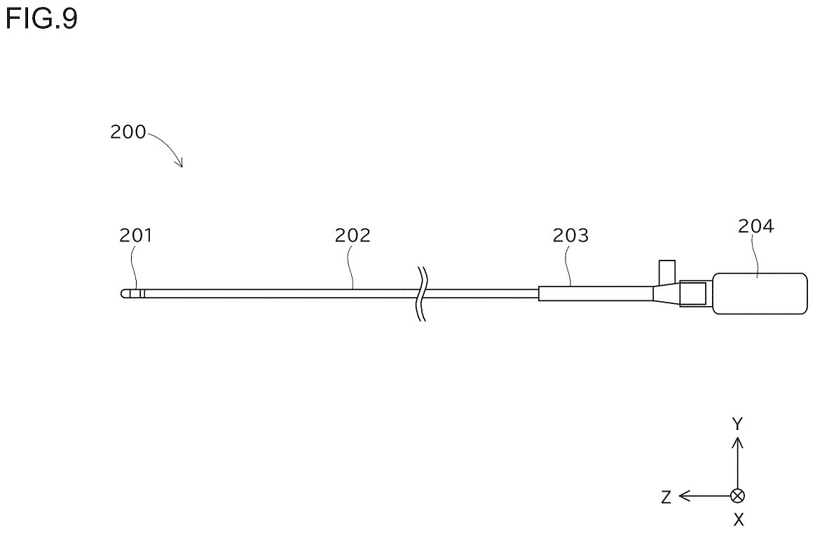

FIG. 9 is an explanatory view schematically illustrating a configuration of the IVUS device 200. The IVUS device 200 is a device for acquiring an image of the interior of a living body lumen, and has an elongated outer shape as a whole. The IVUS device 200 is an example of an imaging device in the claims.

The IVUS device 200 includes a transducer 201, a driving cable 202, a connector 203, and a motor drive 204.

The transducer 201 has an ultrasonic probe (also called an ultrasonic vibrator, piezoelectric body, ultrasonic transmitting/receiving element, or ultrasonic element) that emits ultrasonic waves and receives reflected waves thereof. The motor drive 204 is a device for controlling the rotation of the transducer 201. The motor drive 204 is electrically connected to the imaging console 300 via a cable 50 (FIG. 1). The driving cable 202 is an elongated member and includes a coaxial cable that electrically connects the transducer 201 and the motor drive 204. The connector 203 is a member for connecting the coaxial cable of the driving cable 202 and the motor drive 204.

The imaging console 300 (FIG. 1) is a device that includes circuitry configured to control the IVUS device 200, and generate and display images based on signals received from the IVUS device 200. To be specific, the imaging console 300 may include circuitry configured to output a signal via the cable 50 to cause the transducer 201 in the IVUS lumen 160L to move in the extending direction (Z-axis direction) of the shaft part 110 and to rotate the transducer 201 in the circumferential direction of the shaft part 110 in response to the operation of the adjuster 105. The moving range of the transducer 201 along the extending direction of the shaft part 110 can be set to, for example, a range from the distal end of the second inner shaft 112 to a position separated from the distal end by approximately 100 mm to 200 mm. The circuitry of the imaging console 300 causes the transducer 201 to transmit and receive ultrasonic waves in response to an operation of the operator via an input means. The reflected wave received by the transducer 201 is input to the imaging console 300 via the driving cable 202 and the cable 50. The circuitry of imaging console 300 is configured to generate an image represented by gradation of light and shade corresponding to the intensity of the received reflected wave, and output y the generated image to a display 302 to be displayed thereon. Hereinafter, the image acquired by the IVUS device 200 and displayed on the display 302 is also referred to as a “sensor image”.

### Method of Using the Recanalization Catheter System 10

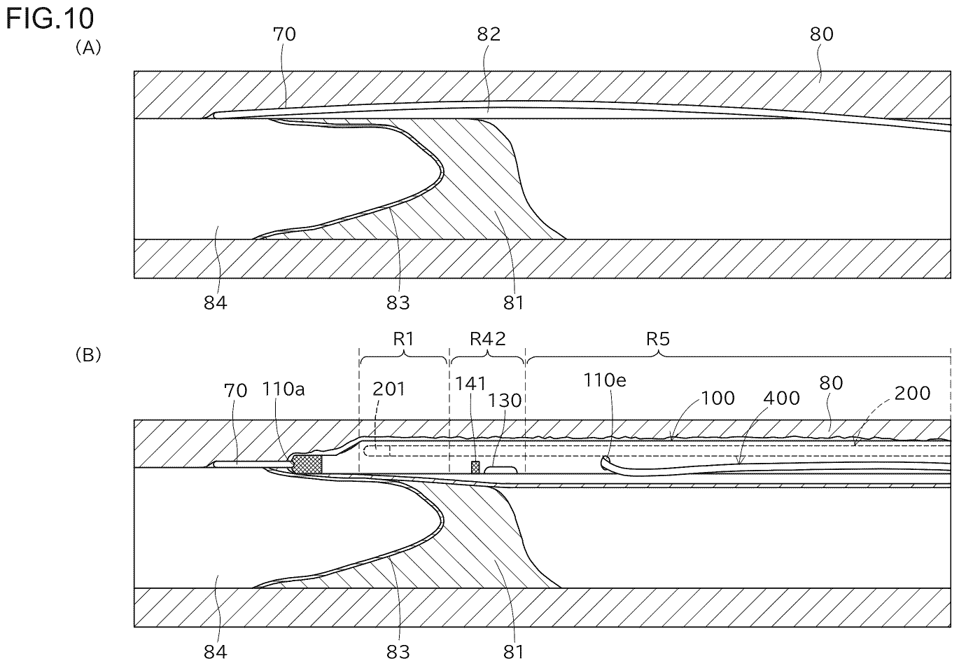

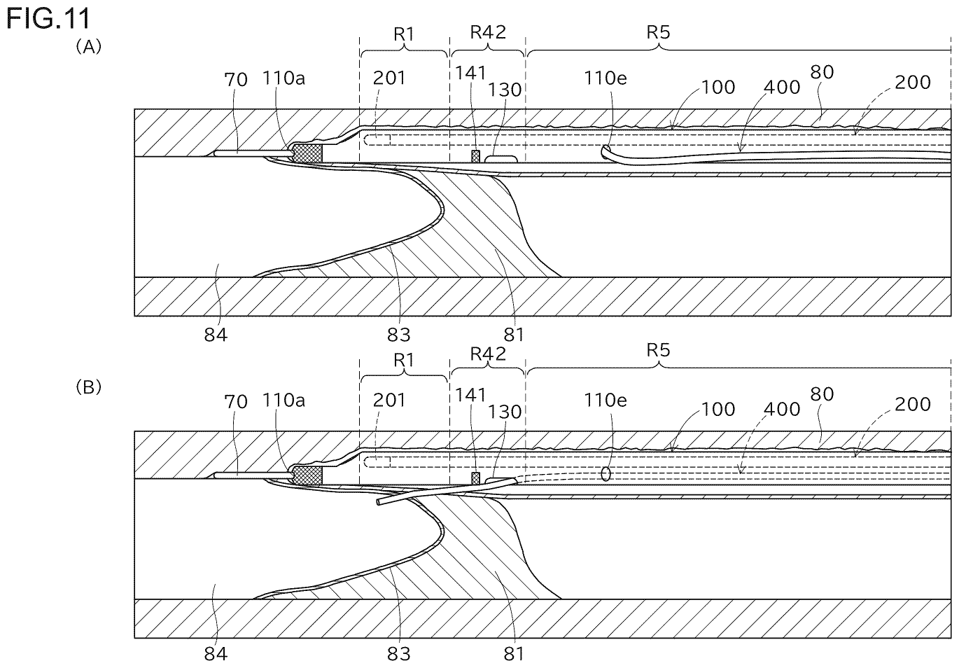

FIGS. 10 and 11 are explanatory views illustrating an example of a method of using the recanalization catheter system 10. FIGS. 10 and 11 illustrate a coronary artery 80 as an example of a living body lumen, a CTO 81 generated in the coronary artery 80, a true lumen 84, a false lumen 82 formed in the intima or subintima of the coronary artery 80 (all dissected lumens other than the true lumen 84 formed by a medical device such as the delivery guide wire 70), and a fibrotic film (plaque) 83 existing between the true lumen 84 and false lumen 82. The fibrotic film 83 may be formed in a fibrous shape on the surface of the CTO lesion.
(A) of FIG. 10 illustrates a state where the delivery guide wire 70 is inserted into the coronary artery 80. In (A) of FIG. 10, the delivery guide wire 70 operated by the operator enters the intima of the coronary artery 80 or forms the false lumen 82 under the intima.
(B) of FIG. 10 illustrates a state where the IVUS catheter 100 is delivered using the delivery guide wire 70. The operator inserts the delivery guide wire 70 into the IVUS catheter 100 by performing the operation described above with reference to FIG. 7. Thereafter, the operator delivers the IVUS catheter 100 to the false lumen 82 using the delivery guide wire 70 as a guide. At this time, the transducer 201 of the IVUS device 200 is disposed in the first region R1 of the IVUS catheter 100.
(A) of FIG. 11 illustrates a state where the positions of the delivered IVUS catheter 100 and IVUS device 200 are adjusted. The operator adjusts each position indicated in the following a1 to a3. The adjustment a2 may be omitted.

### Adjustment a1: Adjustment of the Position Along the Extending Direction of the IVUS Catheter 100

By moving the IVUS catheter 100 along the coronary artery 80, the operator disposes the notch 130 of the IVUS catheter 100 at an optimal position for penetration into the true lumen 84 by the penetration guide wire 400. The adjustment a1 can be performed while checking the position of the coronary artery 80 on the sensor image or the position of the marker 141 on an X-ray image. This adjustment is performed in such a manner that the first region R1 of the IVUS catheter 100 is positioned at the position of the distal end portion of the CTO 81, for example, in order to facilitate confirmation of the position (the position of the distal end portion of the CTO 81) to be penetrated by the penetration guide wire 400 pulled out from the notch 130 by the sensor image. At this time, by referring to the sensor image based on the signal from the transducer 201 positioned in the first region R1, it is possible to accurately adjust the position along the extending direction of the IVUS catheter 100 in such a manner that the first region R1 comes to the position of the distal end portion of the CTO 81 while confirming the position of the distal end portion of the CTO 81.

### Adjustment a2: Adjustment of Orientation Along the Circumferential Direction of the IVUS Catheter 100

The operator adjusts the orientation of the IVUS catheter 100 in such a manner that the notch 130 faces the CTO 81 by rotating the IVUS catheter 100 in the circumferential direction. The adjustment a2 can be performed while confirming the positional relation between the delivery guide wire 70 and the coronary artery 80 on the sensor image.

### Adjustment a3: Adjustment of the Position Along the Longitudinal Direction for the Transducer 201 of the IVUS Device 200

The operator operates the adjuster 105 to move the transducer 201 in such a manner that the position of the transducer 201 becomes a position suitable for observing the penetration of the penetration guide wire 400. The adjustment a3 can be performed while confirming the coronary artery 80 on the sensor image.
(B) of FIG. 11 illustrates a state where the penetration guide wire 400 penetrates a biological tissue. The penetration guide wire 400 is an elongated medical device having a pointed portion at the distal end thereof. The pointed portion of the penetration guide wire 400 is a portion having an arrowhead shape or a wedge shape in which the diameter decreases from the proximal end side toward the distal end side, and enables penetration of a biological tissue by the penetration guide wire 400.

First, the operator removes the delivery guide wire 70. After removal of the delivery guide wire 70, the operator inserts the penetration guide wire 400 into the IVUS catheter 100 by performing the operation described above with reference to FIG. 8. Thereafter, while checking the position of the penetration guide wire 400 on the sensor image, the operator guides the pointed portion of the penetration guide wire 400 to the optimal site of penetration described above. Then, the biological tissue (target tissue) is penetrated using the pointed portion of the penetration guide wire 400, and the distal end of the penetration guide wire 400 is caused to reach the true lumen 84. At this time, the transducer 201 of the IVUS device 200 is disposed in the first region R1 of the IVUS catheter 100 in order to easily confirm the penetrating position by the penetration guide wire 400 by the sensor image.

In this manner, the CTO 81 can be opened by the recanalization catheter system 10. The method described above is merely an example, and the recanalization catheter system 10 can be used in various procedures. For example, the recanalization catheter system 10 is not limited to the approach from the false lumen 82 to the true lumen 84, and may be used when performing an approach to penetrate a CTO from the true lumen 84 on the near side to the true lumen 84 on the distal side.

### A-2. Effects of First Embodiment

As described above, the IVUS catheter 100 of the present embodiment includes the shaft part 110 having the first inner shaft 111 and the second inner shaft 112. The first inner shaft 111 is a cylindrical member having a guide wire lumen 150L into which a guide wire is inserted. The second inner shaft 112 is a cylindrical member having the IVUS lumen 160L into which the IVUS device 200 is inserted, and is disposed side alongside the first inner shaft 111. The second inner shaft 112 is not joined to the first inner shaft 111 in the first region R1 from the first position P1 to the second position P2 positioned further on the proximal end side than the first position P1 in the distal end portion of the shaft part 110, and the second inner shaft 112 is joined to the first inner shaft 111 in the second region R2 that is continuous with the first region R1 and that is positioned further on the proximal end side than the first region R1.

As described above, in the IVUS catheter 100 of the present embodiment, the first region R1 in which the second inner shaft 112 is not joined to the first inner shaft 111 is present at the distal end portion of the shaft part 110. In the first region R1, there is no adhesive for joining the first inner shaft 111 and the second inner shaft 112, and distortion due to thermal welding does not occur in the first inner shaft 111 and the second inner shaft 112. Therefore, when the transducer 201 of the IVUS device 200 is positioned in the first region R1, the transmission and reception of ultrasonic waves on the first inner shaft 111 side by the transducer 201 is not hindered by the presence of the adhesive. Since the first inner shaft 111 is not distorted by welding in the first region R1 as illustrated in FIG. 4, the range (the range of an angle θ1 illustrated in FIGS. 4 and 5) in which the transmission and reception of ultrasonic waves on the first inner shaft 111 side by the transducer 201 of the IVUS device 200 is hindered is narrower than in a portion where the first inner shaft 111 is distorted by welding as in the second region R2 illustrated in FIG. 5. Consequently, according to the IVUS catheter 100 of the present embodiment, interference with the transmission and reception of ultrasonic waves on the first inner shaft 111 side by the transducer 201 due to the joining of the first inner shaft 111 and the second inner shaft 112 can be avoided, and the acquisition of a clearer image by the IVUS device 200 can be achieved.

For example, as described above, when the transducer 201 is disposed in the first region R1 of the IVUS catheter 100 at the time of delivering the IVUS catheter 100 or penetrating with the penetration guide wire 400, by referring to the sensor image based on the signal from the transducer 201 positioned in the first region R1, the position of the distal end portion of the CTO 81 can be accurately confirmed and the IVUS catheter 100 can be delivered to an appropriate position, and the penetrating position by the penetration guide wire 400 and whether the penetration guide wire 400 has reliably penetrated the CTO 81 can be accurately confirmed.

According to the IVUS catheter 100 of the present embodiment, it is possible to improve the flexibility of the shaft part 110 and the operability of the IVUS catheter 100 due to the presence of the first region R1 in which the second inner shaft 112 is not joined to the first inner shaft 111 in the distal end portion of the shaft part 110 which is required to have flexibility but is likely to become hard due to the insertion of the IVUS device 200.

According to the IVUS catheter 100 of the present embodiment, it is possible to reduce the rigidity gap of the shaft part 110 and to improve the kink resistance of the shaft part 110 compared to a conventional configuration in which a large opening (a portion where a resin material is not present) is provided at a position facing the transducer 201 in the first inner shaft in order to achieve the acquisition of a clearer image.

In the IVUS catheter 100 of the present embodiment, the first position P1 is positioned on the proximal end side of the distal end of the second inner shaft 112, and the second inner shaft 112 is joined to the first inner shaft 111 in the third region R3 that is continuous with the first region R1 and that is positioned further on the distal end side than the first region R1. Therefore, according to the IVUS catheter 100 of the present embodiment, the presence of the first region R1 in which the second inner shaft 112 is not joined to the first inner shaft 111 makes it possible to acquire a clearer image, and the presence of the third region R3 which is positioned further on the distal end side than the first region R1 and in which the second inner shaft 112 is joined to the first inner shaft 111 makes it possible to suppress a decrease in the operability of the shaft part 110 due to the provision of the first region R1 which is not joined.

In the IVUS catheter 100 of the present embodiment, the minimum wall thickness value of the shaft part on the guide wire lumen side in the fourth region R4 including the first region R1 is smaller than the minimum wall thickness value of the shaft part on the guide wire lumen side in the fifth region R5 that is continuous with the fourth region R4 and that is positioned further on the proximal end side than the fourth region R4. Therefore, even if the transducer 201 is not positioned in the first region R1, when the transducer 201 is positioned in a portion of the fourth region R4 other than the first region R1, transmission and reception of ultrasonic waves on the first inner shaft 111 side by the transducer 201 are less likely to be hindered than in the case where the transducer 201 is positioned in the fifth region R5. Consequently, according to the IVUS catheter 100 of the present embodiment, by positioning the transducer 201 in the fourth region R4, it is possible to more effectively avoid interference with the transmission and reception of ultrasonic waves on the first inner shaft 111 side (guide wire lumen 150L side) by the transducer 201, and it is possible to achieve the acquisition of a clearer image by the IVUS device 200.

For example, even when the transducer 201 is not positioned in the first region R1 and is positioned in a portion of the fourth region R4 other than the first region R1 at the time of delivering the IVUS catheter 100 or penetrating with the penetration guide wire 400, by referring to the sensor image based on the signal from the transducer 201, the position of the distal end portion of the CTO 81 can be accurately confirmed and the IVUS catheter 100 can be delivered to an appropriate position, and the penetrating position by the penetration guide wire 400 and whether the penetration guide wire 400 has reliably penetrated the CTO 81 can be accurately confirmed.

In the IVUS catheter 100 of the present embodiment, the fourth region R4 includes the first small region R41 and the second small region R42 that is continuous with the first small region R41 and that is positioned further on the proximal end side than the first small region R41, and the minimum wall thickness value of the shaft part on the guide wire lumen side in the second small region R42 is larger than the minimum wall thickness value of the shaft part on the guide wire lumen side in the first small region R41. Therefore, according to the IVUS catheter 100 of the present embodiment, it is possible to increase the rigidity of the shaft part 110 in the second small region R42, and for example, it is possible to secure rigidity when advancing through a bent portion in a blood vessel. It is possible to gradually decrease the rigidity of the shaft part 110 in order of the fifth region R5, the second small region R42, and the first small region R41 which are arranged from the proximal end side to the distal end side, and it is possible to effectively improve the kink resistance of the shaft part 110 by effectively reducing the rigidity gap.

### B. Second Embodiment

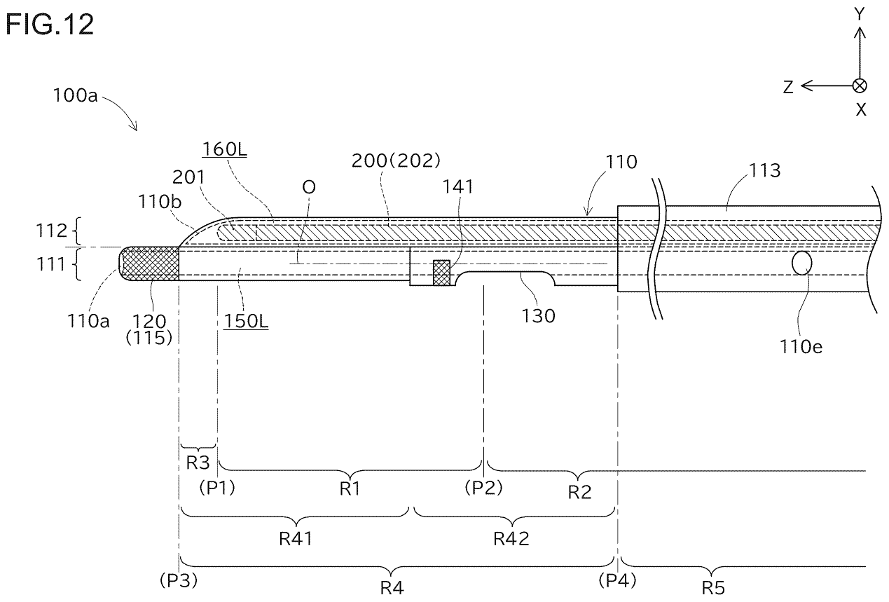

FIG. 12 is an explanatory view schematically illustrating a configuration of an IVUS catheter 100a according to a second embodiment. In the following, among the configurations of the IVUS catheter 100a in the second embodiment, the same configurations as those of the IVUS catheter 100 in the first embodiment described above are denoted by the same reference signs, and description thereof is omitted as appropriate.

The IVUS catheter 100a of the second embodiment is different from the IVUS catheter 100 of the first embodiment in the position of the first region R1 in which the second inner shaft 112 is not joined to the first inner shaft 111. To be specific, in the IVUS catheter 100a of the second embodiment, the first region R1 is set so as to include a part on the distal end side of the notch 130 formed in the shaft part 110. That is, in the IVUS catheter 100a according to the second embodiment, the second inner shaft 112 is not joined to the first inner shaft 111 at a portion of the notch 130 on the distal end side. Therefore, it is possible to acquire a clear image in the vicinity of the notch 130 by the IVUS device 200. Consequently, according to the IVUS catheter 100a of the second embodiment, when the distal end portion of the penetration guide wire 400 is pulled out from the notch 130, a procedure can be performed with reference to a clear image acquired by the IVUS device 200, and the convenience of the procedure can be improved.

### C. Third Embodiment

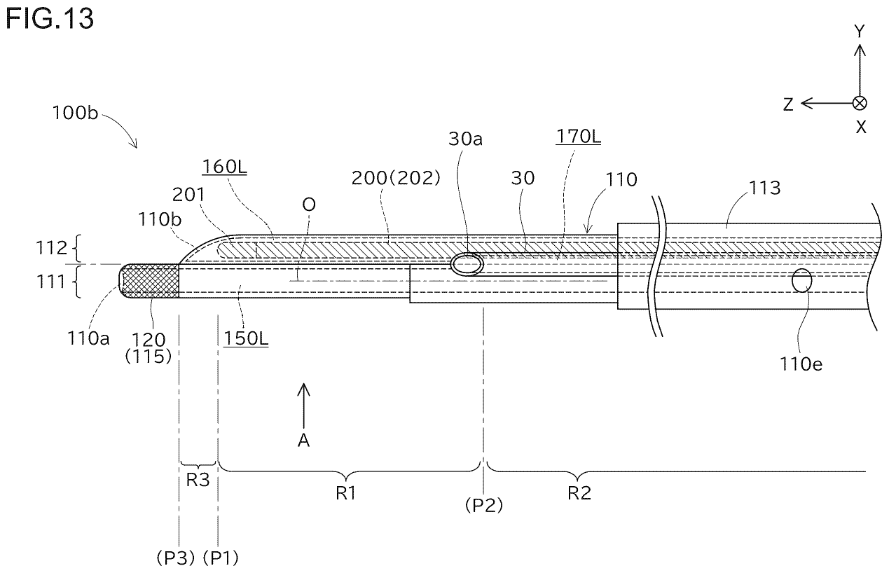

FIG. 13 is an explanatory view schematically illustrating a configuration of an IVUS catheter 100b according to a third embodiment. In the following, among the configurations of the IVUS catheter 100b in the third embodiment, the same configurations as those of the IVUS catheter 100 in the first embodiment described above are denoted by the same reference signs, and description thereof is omitted as appropriate.

The IVUS catheter 100b of the third embodiment is different from the IVUS catheter 100 of the first embodiment in that the IVUS catheter 100b has three lumens. To be specific, the shaft part 110 of the IVUS catheter 100b of the third embodiment includes the first inner shaft 111 having the guide wire lumen (delivery guide wire lumen) 150L into which the delivery guide wire 70 (see FIG. 7) is inserted, the second inner shaft 112 having the IVUS lumen 160L into which the IVUS device 200 is inserted, and a third inner shaft 30 having a guide wire lumen (penetration guide wire lumen) 170L into which the penetration guide wire 400 (see FIG. 8) is inserted. The third inner shaft 30 is an example of a third shaft in the claims.

The third inner shaft 30 is a substantially cylindrical member. The distal end of the third inner shaft 30 is positioned on the proximal end side of the distal end of the first inner shaft 111 and the distal end of the second inner shaft 112. A distal end third opening 30a that allows the guide wire lumen 170L to communicate with the outside is formed at the distal end of the third inner shaft 30. The first inner shaft 111, the second inner shaft 112, and the third inner shaft 30 are arranged alongside in a state where the extending directions thereof are parallel to one another, and are accommodated in the inner space of the outer shaft 113. In the present embodiment, the notch 130 is not formed in the first inner shaft 111.

Also in the IVUS catheter 100b of the third embodiment, similarly to the IVUS catheter 100 of the first embodiment, the second inner shaft 112 is not joined to the first inner shaft 111 in the first region R1 from the first position Pl to the second position P2 positioned further on the proximal end side than the first position Pl in the distal end portion of the shaft part 110, and the second inner shaft 112 is joined to the first inner shaft 111 in the second region R2 that is continuous with the first region R1 and that is positioned further on the proximal end side than the first region R1. Therefore, according to the IVUS catheter 100b of the third embodiment, interference with the transmission and reception of ultrasonic waves on the first inner shaft 111 side by the transducer 201 due to the joining of the first inner shaft 111 and the second inner shaft 112 can be avoided, and the acquisition of a clearer image by the IVUS device 200 can be achieved.

Also in the IVUS catheter 100b of the third embodiment, similarly to the IVUS catheter 100 of the first embodiment, the first position Pl is positioned on the proximal end side of the distal end of the second inner shaft 112, and the second inner shaft 112 is joined to the first inner shaft 111 in the third region R3 that is continuous with the first region R1 and that is positioned further on the distal end side than the first region R1. Therefore, according to the IVUS catheter 100b of the third embodiment, the presence of the first region R1 in which the second inner shaft 112 is not joined to the first inner shaft 111 makes it possible to acquire a clearer image, and the presence of the third region R3 which is positioned further on the distal end side than the first region R1 and in which the second inner shaft 112 is joined to the first inner shaft 111 makes it possible to suppress a decrease in the operability of the shaft part 110 due to the provision of the first region R1 which is not joined.

### D. Modifications

The technique disclosed herein is not limited to the embodiments described above, and various modifications can be made within a scope that does not depart from the gist thereof. For example, the following modifications can be made.

The configurations of the recanalization catheter system 10 and the devices such as the IVUS catheter 100 constituting the recanalization catheter system 10 in the above-described embodiments are merely examples, and can be variously modified. For example, in the above-described embodiments, the third region R3 in which the second inner shaft 112 and the first inner shaft 111 are joined to each other is present on the distal end side of the first region R1 in which the second inner shaft 112 is not joined to the first inner shaft 111, but the third region R3 may not be present, and the distal end of the second inner shaft 112 may not be joined to the first inner shaft 111.

In the above-described embodiments, the minimum wall thickness value of the shaft part on the guide wire lumen side in the second small region R42 is larger than the minimum wall thickness value of the shaft part on the guide wire lumen side in the first small region R41, but the magnitude relation between the two may be reversed, or the two may be the same. In the above-described embodiments, the minimum wall thickness value of the shaft part on the guide wire lumen side in the fourth region R4 is smaller than the minimum wall thickness value of the shaft part on the guide wire lumen side in the fifth region R5, but the magnitude relation between the two may be reversed, or the two may be the same.

In the above-described embodiments, the first inner shaft 111 and the second inner shaft 112 are formed of a single cylindrical body from the distal end to the proximal end, but the first inner shaft 111 and/or the second inner shaft 112 may have a configuration in which a plurality of cylindrical bodies arranged in the extending direction are joined to each other. In the above-described embodiments, the first inner shaft 111 and the second inner shaft 112 are covered by the outer shaft 113, but the outer shaft 113 may be omitted.

In the above-described embodiments, the notch 130 is formed in the IVUS catheter 100, but the notch 130 may not be formed. In the above-described embodiments, the raised portion 152 and the boundary wall 153 are formed in the branching part 150 of the IVUS catheter 100. However, the raised portion 152 and/or the boundary wall 153 may not be formed. In the above-described embodiments, the IVUS catheter 100 is provided with the branching lumen 150Lb branching from the guide wire lumen 150L. However, the branching lumen 150Lb may not be provided.

In the above-described embodiment, the IVUS device 200 is used as the imaging device, but another imaging device such as an optical coherence tomography (OCT) device or a camera may be used instead of the IVUS device 200.

In the above-described embodiments, the recanalization catheter system 10 is a system for using the penetration guide wire 400. However, the recanalization catheter system 10 may be configured as a system for opening a CTO with the use of a plasma guide wire for performing ablation of a biological tissue using plasma without using the penetration guide wire 400. The recanalization catheter system 10 may also be used in other ways not described above. For example, the recanalization catheter system 10 may be used for a blood vessel (for example, a cerebral blood vessel or the like) other than a coronary artery, may be used in a living body lumen other than a blood vessel, and may be used for a treatment or examination other than the opening of a CTO.

1. A catheter comprising a shaft part having:
a cylindrical first shaft having a guide wire lumen into which a guide wire is inserted; and
a cylindrical second shaft having an imaging lumen into which an imaging device for acquiring an image of an interior of a living body lumen is inserted, the second shaft being disposed alongside the first shaft, wherein
in a first region from a first position at a distal end portion of the shaft part to a second position further on a proximal end side than the first position, the second shaft is not joined to the first shaft, and
in a second region that is continuous with the first region and that is further on the proximal end side than the first region, the second shaft is joined to the first shaft.

2. The catheter according to claim 1, wherein a minimum value of a wall thickness of the shaft part at a position of the guide wire lumen in a fourth region that includes the first region in the shaft part is smaller than a minimum value of the wall thickness of the shaft part at a position of the guide wire lumen in a fifth region that is continuous with the fourth region and that is positioned further on the proximal end side than the fourth region.

3. The catheter according to claim 2,
wherein the fourth region in the shaft part includes a first small region and a second small region that is continuous with the first small region and that is positioned further on the proximal end side than the first small region, and
wherein a minimum value of the wall thickness of the shaft part at a position of the guide wire lumen in the second small region is larger than a minimum value of the wall thickness of the shaft part at a position of the guide wire lumen in the first small region.

4. The catheter according to claim 3,
wherein the first position is positioned on a proximal end side of a distal end of the second shaft, and
wherein the second shaft is joined to the first shaft in a third region that is continuous with the first region in the shaft part and that is positioned further on a distal end side than the first region.

5. The catheter according to claim 4, wherein
the first shaft includes a notch communicating with the guide wire lumen at a position on a proximal end side of a distal end in the first shaft, and
wherein the first region in the shaft part includes at least a part of the notch.

6. The catheter according to claim 5, further comprising a cylindrical third shaft having a guide wire lumen into which a guide wire is inserted.

7. The catheter according to claim 5,
wherein the second region in the shaft part includes part of the notch.

8. The catheter according to claim 1,
wherein the first position is positioned on a proximal end side of a distal end of the second shaft, and
wherein the second shaft is joined to the first shaft in a third region that is continuous with the first region in the shaft part and that is positioned further on a distal end side than the first region.

9. The catheter according to claim 1,
wherein a notch communicating with the guide wire lumen is formed at a position on a proximal end side of a distal end in the first shaft, and
wherein the first region in the shaft part includes at least a part of the notch.

10. The catheter according to claim 9,
wherein the second region in the shaft part includes part of the notch.

11. The catheter according to claim 9, further comprising a marker in the first shaft, the marker being closer to the distal end portion of the shaft than the notch.

12. The catheter according to claim 1, further comprising a cylindrical third shaft having a guide wire lumen into which a guide wire is inserted.

13. The catheter according to claim 12, wherein a distal end of the third shaft is positioned further on the proximal end side than a distal end of the second shaft and the distal end of the third shaft is in the first region.

14. The catheter according to claim 1, further comprising a marker in the first shaft.

15. The catheter according to claim 14, wherein the marker is in the first region.

16. The catheter according to claim 14, wherein the marker is in the second region.
